(19) United States
(12) Patent Application Publication
(10) Pub. No.: US 2022/0117548 A1
(43) Pub. Date: Apr. 21, 2022

(54) SYSTEM AND METHODS FOR CUSTOM ADJUSTMENTS OF A LEARNING SYSTEM ACCORDING TO AN INDIVIDUAL'S COGNITION LEVEL

(71) Applicant: TABI learning Technologies Ltd., Ashdod (IL)

(72) Inventor: David BOTRASHVILI, Ashdod (IL)

(21) Appl. No.: 17/504,164

(22) Filed: Oct. 18, 2021

Related U.S. Application Data
(60) Provisional application No. 63/093,356, filed on Oct. 19, 2020.

(51) Int. Cl. A61B 5/00; G06F 3/01; A61B 5/16; G09B 17/00

(52) U.S. Cl. CPC A61B 5/486; G06F 3/015; G09B 17/003; A61B 5/168; A61B 5/163; G06F 3/013

(57) ABSTRACT

The present invention provides a method and system for improving learning capabilities and concentration.

### CROSS REFERENCE TO RELATED APPLICATIONS

This application claims the benefit of priority of U.S. Provisional Patent Application No. 63/093,356 filed Oct. 19, 2020, the contents of which are incorporated herein by reference in its entirety.

### FIELD OF THE INVENTION

The present invention relates in general to learning systems, in particular to their custom and automatic adjustment in accordance to an individual's specific cognition level for improving the overall focus, learning ability, reading and other related matters of the individual.

### BACKGROUND OF THE INVENTION

Many individuals have difficulty learning. In fact, ever since smartphones have come into widespread use among young people, the proportion of children who have had serious difficulty focusing while studying has increased considerably. The proportion of children with learning difficulties has reached 50% (according to certain definitions of learning difficulties) and these numbers have unfortunately been constantly growing.

The way that individuals process words is an essential part of life. When examining the circumstantial model that leads to a diagnosis of dyslexia, visual stress is the element that is most ignored. The spectrum of learning disabilities worldwide is constantly increasing, exceeding the 40% mark, due to visual overload that constitutes the primary obstacle on the path to scholastic success.

Various attempts have been made to improve learning capabilities and concentration during reading and learning.

In a study conducted by Prof. Stanislas Dehaene from the College de France, regarding the ramifications of writing versus typing, it was proven that during the writing process specifically, the brain closes a neural circuit which develops the cognitive and emotional skills of the student. In a study conducted regarding the quantity of paper used in educational institutions, it was discovered that 87.5% of the paper products used by schools every year are thrown into the garbage, instead of not using paper at all, for a better, more ecological society.

Prof. John Stein from Oxford University showed that a yellow background, adapted to the learning environment, has a significantly positive impact on magnocellular cells in all populations with learning disabilities and attention deficit disorders. Technology is the obvious tool for overcoming learning challenges in the 21^{st} century, yet in practice, populations with learning disabilities still use narcotics as a means of achieving a cognitive-sensory balance. Prof. Peter Barret of Oxford University showed that creating a “smart classroom” without pedagogical intervention raised the success rates among students by 55% on average. The changes made in the classroom were: structure of the classroom, the technical materials, dress code, furniture, color of the walls, lighting and temperature. The problem: Sensory information undergoes a conscious processing process. In the event of a learning disability, information received through the eyes leads to a lack of sensory processing that causes terrible visual overload, headaches, and fatigue, which prevent the students from performing basic tasks such as reading and writing without mistakes. The amount of blood that flows to the brain is several times higher in comparison with a normative person, which causes a sense of quick, undefined exhaustion. For those with an attention deficit disorder, the situation is even more severe, because one of the wavelengths that penetrates through the eye is processed erroneously by the brain, causing their vision to be foggy, move and vibrate, which does not allow them to think, concentrate or focus on any task.

Educational institutions need to create equal opportunities for each student in a universal classroom with a heterogenic population. On the other hand, there are not enough appropriate tools to address these needs. Teachers do not have the ability to identify/analyze the student's attention span or emotional situation in real time, so they fail to develop a creative response in real time that could promote empowering, compassionate communication. The 21^{st} century's students, possess intuitive learning skills that are not appropriate for the old-style methods of teaching information only, especially considering the dramatic increase in students with learning disabilities. Paper products create chaos when students with learning disabilities attempt to acquire organizational skills, and they become useless in future interactive learning environments, in addition to their very negative impact on the ecological system. Technology is a powerful tool for advancing and improving learning skills. Yet in practice, the technology has not succeeded in minimizing the distractions and creating a personalized space to deal with scholastic challenges. For this very reason, certain embodiments of the invention were created.

Accordingly, a need exists for an improved system and method for improving literary capabilities of individuals.

### SUMMARY OF INVENTION

In a first aspect, the present invention provides a method for automatic adapting a literacy system for a specific individual, the method comprising the steps of: (a) after activation of the system by said individual, monitoring said individual's attentiveness during/while usage thereof; (b) determining individual's attentiveness level; and (c) providing biofeedback and/or adjusting specific conditions of said literacy system according to the determined individual's attentiveness level, wherein: if the individual's attentiveness level is within a predefined threshold range, no adjustments are needed; and if the individual's attentiveness level is outside said predefined threshold range, specific conditions are adjusted until the individual's attentiveness level is determined to be within said predefined threshold range.

In a second aspect, the present invention provides a personalized automatic-adapting cognitive process system designed to improve individual's focus/attention and literacy capabilities, the system comprising: (a) a sensor designed to track said individual's sense(s); and (b) a literacy and cognitive-tracking system comprising a computing system comprising: a processor, a memory, an artificial intelligence (AI) module and a display screen, wherein: the sensor is designed to monitor individual's sense(s) and transmit data to the AI module; the AI module, compares said transmitted data to pre-stored big data, and determines based thereon the individual's attentiveness level; and the AI module determines whether modifications in the display screen and/or a biofeedback are needed (or not) in order to improve individual's focus/attention.

### BRIEF DESCRIPTION OF DRAWINGS

Various embodiments are herein described, by way of example only, with reference to the accompanying drawings, wherein:

FIGS. 1A-1D are photos of one possible configuration of a system of the invention: FIG. 1A is of a rolled up system; FIG. 1B is of an open system with an unfolded screen; FIG. 1C is of a detachable screen and a standalone image detector; and FIG. 1D is an illustration of a system comprising an angled or curved laptop display screen;

FIG. 2 is an illustration of a visually advantageous screen in a system of the invention, having white and light-blue lines; and

FIG. 3 is a photo of a screen having a yellow visually advantageous appearance.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Today, learning is carried out in similar manner to all students, whereas students that have been identified has suffering from learning impairment learn in dedicated facilities or classes with personalized tutoring. However, even in such personalized tutoring, the learning and reading material is essentially similar to that used in standard teaching. Moreover, many students that have not been identified as suffering from some sort of learning deficiency, do not receive proper conditions for learning and reading. In addition, even “normal” students/individuals experience temporary learning/reading deficiencies, e.g., when they are tired or bored, or after a long class, etc.

Accordingly, the present invention is aimed at providing a personalized automatic adjusted learning and reading environment that provide the user with a unique and personalized learning experience that is constantly adjusted according to the user's cognition state. The system and method according to the present invention are based on artificial intelligence (AI), monitoring of the user's cognition's condition, and subsequent adaptation of the sensory surrounding conditions, e.g. visual and sound, for improving user's concentration and focus.

The Sensory Adapted Learning (SAL) platform of the present invention is an AI-based interactive learning platform that creates a personalized brain-eye interface to deal with visual disorder stemming from sensory processing disorder, or due to a cognitive problem processing sensory information. A didactic operating system that gives every student an equal opportunity and creates a personally adapted, organized and focused learning environment for any student dealing with any type of learning disability. In certain embodiments, a system equipped with the SAL platform replaces today's backpack/computer with a multi-purpose computer designed, e.g. as a rolling screen, and functioning as both a screen, a laptop computer and a personalized learning space, and including features such as: typing, writing, reading, control and need-based supervision. In certain embodiments the invention provides a personalized “smart classroom” facilitating compassionate communication for both the educator and the students, and creates an interactive learning experience based on the visual code for cognitive order and focus.

Accordingly, in a first aspect, the present invention provides a method for automatic adapting a literacy system for a specific individual, the method comprising the steps of: (a) after activation of the system by said individual, monitoring said individual's attentiveness during/while usage thereof; (b) determining individual's attentiveness level; and (c) providing biofeedback and/or adjusting specific conditions of said literacy system according to the determined individual's attentiveness level, wherein: if the individual's attentiveness level is within a predefined threshold range, no adjustments are needed to be effected in the literacy system; and if the individual's attentiveness level is outside said predefined threshold range, specific conditions are adjusted until the individual's attentiveness level is determined to be (back) within the predefined threshold range.

In certain embodiments, the present invention provides a method for automatic adjustment to different cognition levels system for a specific individual, the method comprising the steps of: (a) after activation of the system by said individual, monitoring said individual's attentiveness during/while usage thereof; (b) determining individual's attentiveness level; and (c) providing biofeedback and/or adjusting specific conditions that will be (cognitively) personalized for the specific individual preferences of said system according to the determined individual's attentiveness level, wherein: if the individual's attentiveness level is within the predefined threshold range, no adjustments are needed; and if the individual's attentiveness level is below or above said predefined threshold range, specific conditions are adjusted until the individual's attentiveness level is determined to be within said predefined threshold range.

The term “individual's attentiveness” as used herein throughout the application includes an individual cognitive condition and includes whether the individual is dozing away, daydreaming, concentrated/focused, bored, stressed, etc.

The terms “literacy” and “literary” as used herein interchangeably refer to reading, writing, learning, and other related matters associated with learning. Specifically, they refer to learning by electronic means, such as a computer and tablet that enable adjustment of the learning conditions according to the method of the invention.

In certain embodiments, the method according to the invention further comprises a preliminary system-calibration step designed to obtain preliminary data of the specific user using the literacy system for enabling a more accurate adjustment of the literacy system to the specific user. Such preliminary system-calibration steps comprise any one or combination-of the following steps: (i) asking said individual questions regarding: age, sex, ethnicity, native language and/or origin, or any combination thereof; (ii) asking said individual questions regarding specific physical conditions, such as color blindness, dyslexia, ADD, ADHD, Autism, visual impairment, other learning disabilities and spectrum thereof, etc.; and/or (iii) modifying visual characteristics on a display screen, such as brightness, background, color, spacing, font size, shape and color, etc., or any combination thereof, and receiving feedback from the user—either directly or by monitoring individual's eye reaction.

In certain embodiments of the method according to any of the embodiments above, the step of monitoring individual's attentiveness means monitoring any one of: individual's eye movement; page-refreshment/reading speed; and typing speed; or any combination thereof.

In certain embodiments of the method according to any of the embodiments above, the step of providing biofeedback and/or adjusting specific conditions means any one of: modifying visual characteristics on a display screen, such as brightness, background, color, word- and line-spacing, font size, shape and color, etc., or any combination thereof, and subsequently monitoring individual's eye reaction to such modification(s).

In certain embodiments of the method according to any of the embodiments above, the step of providing biofeedback and/or adjusting specific conditions means vocal effects, such as relaxing music, praising sentences, etc.

In specific embodiments, the present invention provides a method for automatic adapting a literacy system for a specific individual, the method comprising the steps of: (a) after activation of the system by said individual, monitoring said individual's attentiveness during/while usage thereof by monitoring any one of: individual's eye movement; page-refreshment/reading speed; and typing speed; or any combination thereof; (b) determining individual's attentiveness level; and (c) providing biofeedback and/or adjusting specific conditions of said literacy system according to the determined individual's attentiveness level by any one of: modifying visual characteristics on a display screen, such as brightness, background, color, word- and line-spacing, font size, shape and color, etc., or adjusting vocal effects, such as relaxing music, praising sentences, or any combination thereof, and subsequently monitoring individual's eye reaction to such modification(s), wherein: if the individual's attentiveness level is within a predefined threshold range, no adjustments are needed; and if the individual's attentiveness level is outside said predefined threshold range, specific conditions are adjusted until the individual's attentiveness level is determined to be within said predefined threshold range.

In specific embodiments, the above method further comprises a preliminary system-calibration step, comprising any one of: (i) asking said individual questions regarding: age, sex, ethnicity, native language and/or origin, or any combination thereof; (ii) asking said individual questions regarding specific physical conditions, such as color blindness, dyslexia, ADD, ADHD, visual impairment, other learning disabilities and spectrum thereof, etc.; and/or (iii) modifying visual characteristics on a display screen, such as brightness, background, color, spacing, font size, shape and color, etc., or any combination thereof, and receiving feedback from the user—either directly or by monitoring individual's eye reaction, or any combination thereof.

In a second aspect, the present invention provides a personalized automatic-adapting cognitive process system designed to improve individual's focus/attention/awareness and literacy capabilities, the system comprising: (a) at least one sensor, e.g., a camera, designed to track said individual's sense(s); and (b) a literacy and cognitive-tracking system comprising a computing system comprising: a processor, a memory, an artificial intelligence (AI) module and a display screen, wherein: the sensor is designed to monitor individual's sense(s) and transmit data to the AI module; the AI module, compares said transmitted data to pre-stored big data, and determines based thereon the individual's attentiveness level; and the AI module determines whether modifications in the display screen and/or a biofeedback are needed (or not) in order to improve individual's focus/attention/awareness.

In certain embodiments, the system is designed to monitor the individual's awareness/concentration/focus level. The monitoring can be continuous or in predefined time intervals determined according to need and/or according to the identified focus level. Accordingly, in certain embodiments, the computing system is designed to receive data from the at least one sensor and analyze same in real-time to determine the individual's awareness/concentration/focus level and if the computing system determines that the individual is not focused (i.e. the focus level is outside a predefined range), it effects modifications in the display (in term of color changing, frequency, font size and shape, etc.) and/or effect sound effects, until it identifies that the individual's attention reaches a predefined level. Accordingly, the system may at first monitor the individual's focus level every, e.g., 1 min., and once it identifies that the individual's focus level is outside the predefined range, it effects such display and/or sound modifications and then monitors the individual's focus level constantly until it identifies the individual's focus level is back in the desired range.

In certain embodiments, the above system is further designed to reduce the individual's inattention, mind distraction, doziness, and/or fatigue, or all, during reading, writing, learning, and answering questions (e.g. during a test).

When an individual is tired, he/she tend to close their eyes, blink a lot and dose-off. Similarly, when an individual is bored, his/her eyes start wondering around for more interesting things. Accordingly, identification of such marks of fatigue or boredom is essential for maintaining the individual's attention and focus. Thus, in certain embodiments of the system of the invention, the sensor designed to track said individual's sense(s) is a sensor or camera designed to track the individual's eyes' movement.

In specific embodiments of the system, the individual's sense is vision, and the system is designed to track said individual's eye(s) movement. In further specific embodiments, the sensor is a camera.

Other signs of boredom or fatigue are slower breathing, increased head movement and touching the face and eyes, as well as reduction in reading and writing speed. Accordingly, in certain embodiments of the system according to any of the embodiments above, the sensor designed to track said individual's sense(s) is a sensor or camera designed to track any one of the individual's: breathing, head position and posture, facial touching, etc. or any combination thereof. In further or alternative embodiments of the system according to any of the embodiments above, the sensor designed to track said individual's sense(s) is a sensor or camera designed to track individual's typing and/or reading speed.

In certain embodiments, the system according to any of the embodiments above further comprises at least one of the following: (i) an input unit, such as a keyboard, touchpad, touchscreen, or smart-pen, or any other accessory that can be used for inputting data into the system by the user; (ii) sensors designed to measure individual's: attention level, visual focus, concentration, brain-eye focus, or doziness, or any combination thereof. Such sensors can be, e.g., camera(s) monitoring individual's eyes' movement, pupil's/oculus's movement and diameter, etc., head posture and tilt, breathing monitor, typing speed sensor, etc, or any combination thereof; and (iii) speakers designed to produce sound(s) as part of a biofeedback to improve individual's focus, attention and awareness.

In specific embodiments of the system according to any of the embodiments above, the screen is flexible, foldable and/or rollable, thereby enabling rolling the screen during storage and carry to save space and protect the screen.

In certain embodiments, the system and method according to any of the embodiments above are designed as an advanced management tool for educators, personalized digital literacy and integrative learning, and can be adapted for any educational institution—from preschool to academia.

In certain embodiments, the system according to any of the embodiments above is comprised of an autonomic computer/tablet that comprises a display with the quality of paper look & feel. In further embodiments, the display is flexible and can be folded as a roll-up screen. In further embodiments, the screen or other components of the system further comprise multi-dimensional sensors for monitoring the user's cognitive state and attention.

The system and method according to any of the embodiments above are very useful for improving literacy capabilities, such as learning, reading, writing, and typing, and for displaying local multiparticipant applications and maximum usage of the learning environment.

As explained, the system according to any of the embodiments above is designed to identify a student's degree of concentration and cognitive focus, using an Artificial Intelligence (AI)-based tracking sensor/camera, in order to personalize the learning environment so that the student can benefit from an organized, focused learning experience. A didactic operating system with unique, multi-purpose hardware, which creates a uniform platform for all future learning content in any learning environment. Certain embodiments implement both a personalized learning environment for each student (with an emphasis on populations with learning disabilities) as well as designing the overall learning environment, in order to create: (a) an interactive learning experience, (b) an organized learning process, and (c) focused thought and vision.

In certain embodiments, the system and method according to any of the embodiments above, is designed to improve literacy capabilities and accessibility of challenged populations, such as learning-impaired individuals.

In certain embodiments, the present invention provides a method and system for custom tailoring visual images and optionally sounds and background sounds for improving literacy capabilities, especially for learning impaired individuals.

A “learning impairment” individual can be determined as such by specialists, before starting to work with the system of the invention. Alternatively, the system of the invention can determine that an individual is suffering from learning impairment (either constant or temporary for a certain period of time during usage of the system). A “learning impairment status” is determined by the system of the invention for an individual, e.g., by a learning impairment status determination module using data received from various sensors and camera(s) associated-with or within the system. The determination of the learning impairment status and degree thereof may be evaluated and adjusted constantly or in predefined intervals, e.g. every 15 sec., every 30 sec., every 1 min., etc., and/or in every change in the average color of the screen, etc. The learning impairment status may include a categorization of the type of learning impairment and its severity (or that there is no learning impairment).

In response to the determination of an individual's learning impairment status and degree, an image modification module adjusts an image or a series of images that are configured to stream to a display screen configured to face the individual. The image modification module is configured to make changes in one or more visual parameters of the image or series of images. The term “visual parameter” is broadly defined to encompass any parameter, such as, but not limited to, color, frequency, brightness, etc., that influences or affects the visual perception of the image(s) shown on the screen to the individual, including also but not limited to the timing or rhythm in which visual images of various types are displayed on the screen or any other parameter described herein. The modification may also include sound effects. The modifications are configured to enhance a sensory/visual experience of the individual, tailored to the learning impairment status of each specific individual. The modifications may be permanent or temporary modifications or may vary in time or may be intermittent, all as necessary for obtaining optimal visual experience for the individual, and in accordance with an algorithm within the computing system. In some implementations, the modifications are made and the new images are provided in the modified form unless or until new data is inputted from the learning impairment status determination module or image detector.

In certain embodiments, the system and method according to any of the embodiments above are configured to adjust the display seen by the individual using the system so as to be so tailored based on information, such as updateable real time information, concerning the learning status of the individual. The learning status of the individual may be determined by the computer system from a number of factors including, but not limited to, data received from a camera that is part of the system. The camera may obtain video data of the individual's face including the individual's eyes. Certain details such as the pupil size of the individual, his or her eye movements, and other data may be used by an algorithm (software forming part of the processing system of the system) to determine the level of attentiveness and other learning parameters of the individual which is used together with other data (including the individual's previous learning status history) to determine the learning status of the individual. Based on that learning status, which may be in the form of an input from a camera to a processing unit, the display screen may adjust the visual images presented to the individual and this adjustment may be constantly revised and updated.

The system according to any of the embodiments above may be designed in any configuration, and can be either integrated with existing components, such as a display screen, keyboard and scroller/mouse, camera(s), etc., or may be a standalone system that includes all the different components. In specific embodiments, when the system is a standalone system, it may be configured as a laptop or as a monitor/touchscreen that can be easily moved from place to place, and may optionally be foldable to reduce its size for easy carrying.

In certain embodiments of the system according to any of the embodiments above, the sensor(s) and camera(s) used thereby are standalone sensor(s) and camera(s) associated with the computing system, e.g. via cables or wirelessly. Alternatively, the sensor(s) and camera(s) may be integrated within the system. For instance, a camera may be paced in the screen (as done in laptop and tablets).

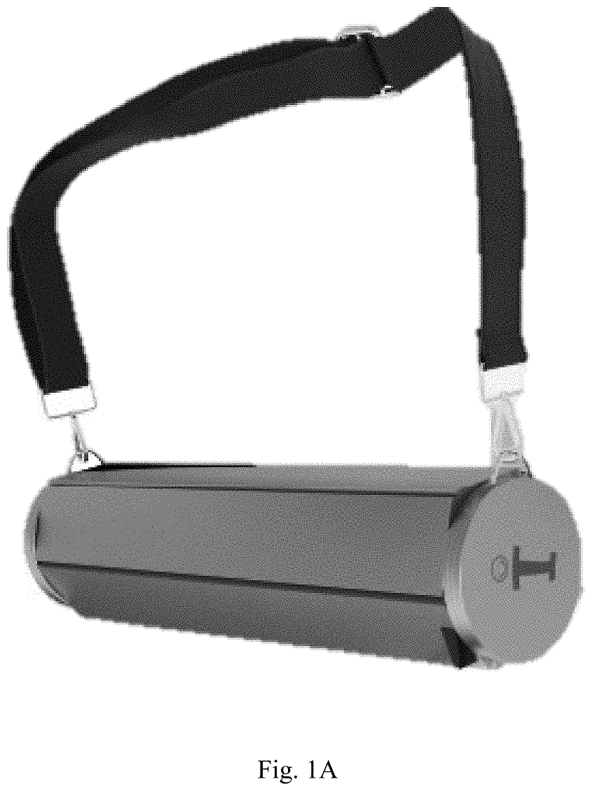

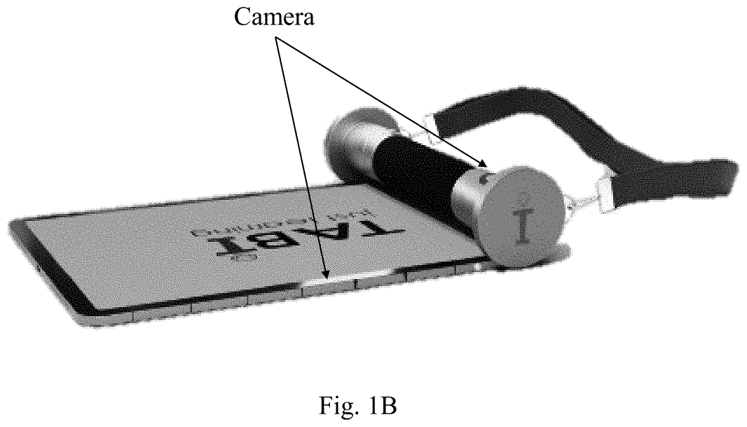

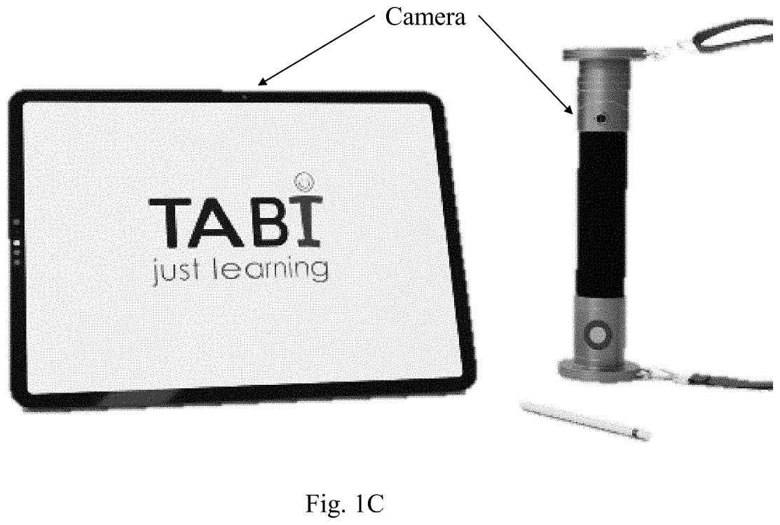

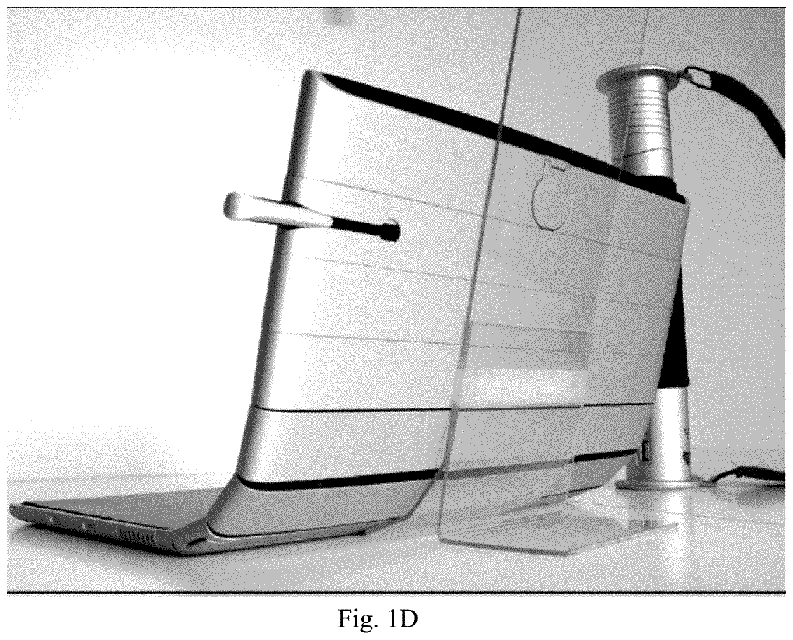

In some embodiments, and as shown in FIGS. 1B & 1C, a camera(s) can be situated on a component configured as an isolated fixed stick and/or physically integrated into the display device itself. The camera(s) can be integrated directly into the computer system itself. The sensor/camera is designed to constantly monitor various parameters of the user. For instance, a camera may be used to monitor user's eye movement, pupil size, pupil diameter, facial movements, movement of the head and other parameters discernable from one or a series of visual images (for example in video) of the individual.

By configuring the camera component as a standalone camera component, in some embodiments, this allows the camera to be focused on the cues that the system needs for determining the status of the learning impairment if any of the individual and allows the system to disregard and not be distracted by other actions taken by the individual. For example, if the camera is integrated in the display device, when the student writes, reads or types, such action may cause the display device containing the camera to move and thereby interfere with the function of the camera, in which case the camera may require fixation algorithm that enable it to maintain focus and disregard any disturbances caused by any such movement thereof.

Another configuration of the system of the invention is as a foldable laptop that opens so as to present a full screen (both halves or all thirds or portions) thus enhancing the visual experience for the visually impaired. In specific versions, this means that both halves or all portions of the laptop, or other device, when fully opened merge into a single screen.

In specific embodiments, the system according to any of the embodiments above is configured as a rolling laptop. For example, as shown in FIGS. 1A-1D, the system includes a substantially cylindrical base or substrate functioning as a camera/sensor and a laptop component fitted on the base. In some versions the whole assembly may include a substantially cylindrical base (which may have end portions that may have wider diameters than the remainder) on which is laid a substantially cylindrical hollow component. The base functions as a camera/sensor component. For example, the base in certain embodiments stands up a standalone stick that has a lens near a top portion incorporating a camera or image detector. The hollow substantially cylindrical portion that fits on the base is the laptop component (processing system and display screen). The laptop component, in some embodiments, may be removed from the base by rolling it off like one would unroll a roll of paper towels off a cardboard hollow cylinder (its base) except that the number of times that the hollow substantially cylindrical (laptop) component revolves around the base in this case is much fewer, such as only approximately two revolutions in some embodiments (although that number can vary widely depending on the thickness of the laptop component, the desired diameter of the assembly, and the length of the laptop component). As shown in FIG. 1D, the rollability of the laptop may be achieved by having the laptop be comprised of sections that can be angled with respect to adjacent sections thereby allowing a curvature to be achieved without detracting from the operability of the screen. In one non-limiting example there can be 8-12 such sections as somewhat apparent from FIGS. 1B & 1D.

In some implementations the laptop component is a computer screen rollable and unrollable into a hollow substantially cylindrical shape that fits onto the base. Once this hollow substantially cylindrical outer shape/portion is unrolled off the base, opens into a computer screen. The computer screen may also have several modes including a first mode in which two or three portions are angled one to the other. For example, one half or one quarter or one fifth or one seventh or one tenth of a length of one dimension of the screen or a portion of a screen or one portion is angled with respect to the portion or alternately each of three portions are angled such that the middle portion is angled at an acute angle with each of the other two portions so as to create a substantially curved overall appearance. One of these embodiments is shown in FIG. 1A. In a second possible mode, the portions are fully opened into a substantially flat screen (FIGS. 1B & 1C). Accordingly, instead of half of the top surface of the opened laptop comprising the display screen as in a standard laptop, the entire or substantially all or approximately 90% or more of the top surface functions as a single computer screen or monitor. The rollable and unrollable component sits on the base until it is rolled off it.

In certain embodiments, the viewable portion of the laptop, i.e. the display screen is wider relative to its total size (in stored position it may be rolled up). This means that it provides a further advantage for learning impaired individuals or students who would normally be more comfortable with a visually expanded surface to look at.

As illustrated in FIG. 1C, the system may also include a stylus or digital writing instrument.

In certain embodiments of the system according to any of the embodiments above, the display screen has a paper-like feeling, e.g. like the feel of the paper of a notebook.

Any one or all of the above structural features of the system of the invention can be used to implement the method according to any of the embodiments above.

In certain embodiment, the method according to the invention comprises some or all of the following steps:

(a) determining a nature of a visual impairment or a learning ability status from among two, three, four or more types of learning disability or impairment; and subsequently displaying learning material visually on a screen to a student/learning impaired individual, wherein the material is presented in one visual form tailored to one particular learning impairment selected from a plurality of visual forms each of which is tailored to a different form of learning impairment (for example amongst a range of visual formats that it can be presented in for learning impaired individuals).

(b) positioning a camera so as to face the student/learning impaired individual while the individual is viewing the screen. The camera is configured to detect or gather date useful for the computing/processing system in determining the extent of and the nature of the learning impairment at each given time. Specifically, the camera is configured to monitor one or more of the following parameters relating to one or more of the following (and/or other features): the size or diameter of the pupils or other parts of the individual's eyes or the changes in such size or diameter, for example the pupils of the individual being reduced, facial expressions, a degree of movement of the head of the individual, a rhythm or timing of the movement of the head or the eyes or the pupils, and angle at which the individual looks at the screen, an amount of time that the individual maintains a direct view of the screen, a number of times that the individual looks away from the screen, a color of the eyes, a number of frequency of blinking, a number or frequency or pattern of facial stress, a determination of any of the above features for one or more of the colors of the spectrum appearing on the display screen and/or any other feature associated with or connected with the level of attentiveness of the individual, and a response of the eye to the visual information appearing on the screen and how and how rapidly the eye changes its response and/or level of attentiveness when the nature of the visual image presented changes. The camera (or other suitable sensor) may thus work to identify, for example in real time, the level of attentiveness of the individual and in general response of the eye to the information that appears on the screen/surface.

In certain embodiments, the camera/sensor is physically separated from the display screen. This allows the camera to more precisely monitor the eyes of the individual looking at the screen. In alternative embodiments, the camera/sensor is position within the display screen. In alternative embodiments, two or more cameras/sensors are used. In specific such embodiments, one camera/sensor may be within the display screen and another camera/sensor is physically separated from the display screen, thereby enabling obtaining even more accurate monitoring and data.

The camera/sensor is designed to forwards any data it collects to the processor within the computing system which analyzes the data in real time and figures out, for example using artificial intelligence, what would be the ideal visual presentation of the screen to be presented to this individual. The processor will take into consideration the appearance of parts of the screen surrounding the text or images that the reader focuses on.

In certain embodiments of the method according to the invention, the steps of (i) monitoring the eyes, face and/or head (or any other parameter) of the individual after presenting custom tailored images based on an algorithm and (ii) then modifying the visual presentation of images based on an updated assessment of the individual's response, are repeated constantly as long as the individual is using the system. In one particular example, if the images on the screen are presented lighter to accommodate the sensitivity of the individual based on the number of the times the individual does something or how long it takes to do something, if after the new images are presented, this parameter is found to change, the lightness of the images can be further modified.

(c) instructing, by the processor, with the support of software, an image generator to modify the visual image based on various factors including, but not limited to, one or a combination of the following: brightness, contrast, amount of whiteness, size of letters, distribution of letters, size of the active portion of the screen, shading, layout, spacing and/or other visual cues.

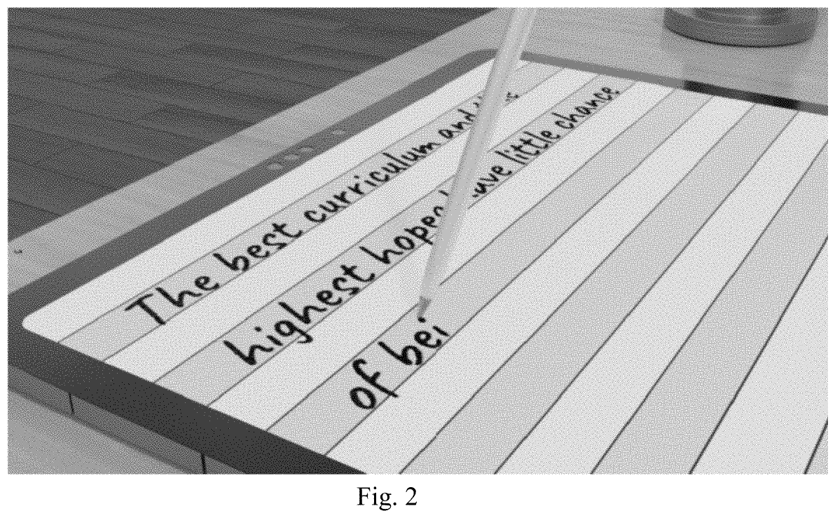

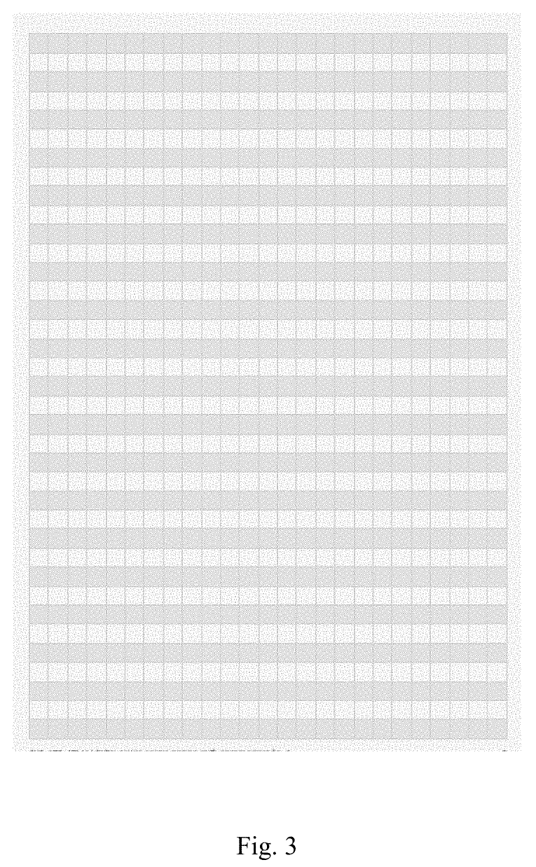

FIGS. 2 & 3 show examples of a screen whose layout and color has been configured for a particular status of learning impairment. For example, the processor may instruct the image generator to change the entire background and the text according to the visual stress or the visual disruption the individual is suffering from. In one non-limiting example with respect to screens presenting reading material: (a) for visually impaired—the individual will enjoy and may be provided with light yellow background, with 100% contrast of the entire lines in the page (see illustrated in FIG. 3); (b) for learning disability—the individual will enjoy and may be provided with a light yellow background with light blue separation lines, with very low contrast (less than 20%) of the lines on the page or screen (see illustrated in FIG. 2); (c) for an individual with a visually processing disorder—the individual will enjoy and may be provided with personally adapted light background, depending on the color found to be missing in his visual spectrum, and the writing line vs. separation line will be in higher contrast, which the lines remain in very low contrast (less than 20%) (see illustrated in FIG. 2); (d) for ADHD (attention deficit hyperactivity disorder) individuals—the individual will enjoy and may be provided with light yellow background with possibility to select dark/light writing line, vs. light/dark separation line, with 10% contrast in lines (see illustrated in FIG. 3). In certain embodiments, the processor instructs the display device to display the learning material visually on the same screen to the same visually impaired individual but as a modified visual image incorporating the changes that were made.

While the above is configured for screens designed for reading, for screens that are designed for writing, the processor may instruct the image generator to create a personalized writing platform tailored to each disability/difficulty that optimizes the brain-eye connection.

In some embodiments, the modifications made by the image modification module may be tailored to the learning impairment status. For example, for an individual determined to have dyslexia, the modification module may generate a change in the spacing between words and/or the spacing between lines. This can be applied across the board to all sentences being presented on the screen. In a different case, if the individual is determined to be visually impaired, the words can be enlarged for example 3-4 times their regular size and/or the contrast can be amplified. For example, as seen in FIG. 2, there is a white or off-white separation stripe between the blue lines on which text appears, the contrast between the separation stripe and the strip used for writing text can be quantified and for example for visually impaired individuals the contrast can be adjusted to be 100%, whereas for example for dyslexia the contrast can be modified to be 30% contrast. Thus, the contrast parameter, the lettering size, the spacing between words and the spacing between lines are non-limiting examples of four visual parameters that the algorithm (or processing system or image modification generator or image modification module) may modify in response to a determination of the learning impairment status of the individual.

The processes (methods) and systems, including components thereof, herein have been described with exemplary reference to specific hardware and software. The methods have been described as exemplary, whereby specific steps and their order can be omitted and/or changed by persons of ordinary skill in the art to reduce these embodiments to practice without undue experimentation. The methods and systems have been described in a manner sufficient to enable persons of ordinary skill in the art to readily adapt other hardware and software as may be needed to reduce any of the embodiments to practice without undue experimentation and using conventional techniques.

While the methods above may be implemented using the hardware/system described above, the methods may also be implemented using other hardware and components suitable to implement any embodiment of the method.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention.

It was found that unique appearances generated on the display of the system have decisive impact on student's ability to focus during reading and writing and create visual stimulus that encourages reading and writing. An examination by clinical psychologists showed that the visual structure of certain embodiments of the invention lowers student's anxiety levels during tests by about 30 to about 50% and improves grade averages by 10-20%. When examining visual overload challenges it was found that the system has a decisive impact on the degree of thought and concentration of students with learning disabilities and attention deficit disorders.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for automatic adapting a literacy system for a specific individual, the method comprising the steps of:
a) after activation of the literacy system by said individual, monitoring said individual's attentiveness during/while usage thereof;
b) determining individual's attentiveness level; and
c) providing biofeedback and/or adjusting specific conditions of said literacy system according to the determined individual's attentiveness level, wherein:
if the individual's attentiveness level is within a predefined threshold range, no adjustments are needed; and
if the individual's attentiveness level is outside said predefined threshold range, specific conditions are adjusted until the individual's attentiveness level is determined to be within said predefined threshold range.

2. The method of claim 1, further comprising a preliminary system-calibration step, comprising:
asking said individual questions regarding: age, sex, ethnicity, native language and/or origin, or any combination thereof;
asking said individual questions regarding specific physical conditions, such as color blindness, dyslexia, ADD, ADHD, visual impairment, other learning disabilities and spectrum thereof, etc.; and/or
modifying visual characteristics on a display screen, such as brightness, background, color, spacing, font size, shape and color, etc., or any combination thereof, and receiving feedback from the user—either directly or by monitoring individual's eye reaction.

3. The method of claim 1, wherein step (c) of providing biofeedback and/or adjusting specific conditions is personalized of said literacy system according to the determined individual's attentiveness level of monitoring individual's attentiveness means monitoring any

4. The method of claim 1, wherein monitoring individual's attentiveness means monitoring any one of: individual's eye movement; page-refreshment/reading speed; and typing speed; or any combination thereof.

5. The method of claim 1, wherein providing biofeedback and/or adjusting specific conditions means any one of: modifying visual characteristics on a display screen, such as brightness, background, color, word- and line-spacing, font size, shape and color, etc., or any combination thereof, and subsequently monitoring individual's eye reaction to such modification(s).

6. The method of claim 1, wherein providing biofeedback and/or adjusting specific conditions means vocal effects, such as relaxing music, praising sentences, etc.

7. A personalized automatic-adapting cognitive process system designed to improve individual's focus/attention and literacy capabilities, the system comprising:
a) a sensor designed to track said individual's sense(s); and
b) a literacy and cognitive-tracking system comprising a computing system comprising: a processor, a memory, an artificial intelligence (AI) module and a display screen, wherein:
the sensor is designed to monitor individual's sense(s) and transmit data to the AI module;
the AI module, compares said transmitted data to pre-stored big data, and determines based thereon the individual's attentiveness level; and
the AI module determines whether modifications in the display screen and/or a biofeedback are needed (or not) in order to improve individual's focus/attention.

8. The system of claim 7, wherein said individual's sense is vision, and the system is designed to track said individual's eye(s) movement.

9. The system of claim 8, wherein said sensor is a camera.

10. The system of claim 7, further comprising an input unit, such as a keyboard, touchpad, touchscreen, or smart-pen.

11. The system of claim 7, further comprising sensors designed to measure individual's:
attention level, visual focus, concentration, brain-eye focus, or doziness, or any combination thereof.

12. The system of claim 7, further comprising speakers designed to produce sound(s) as part of a biofeedback.

13. The system of claim 7, wherein said screen is flexible, foldable and/or rollable.
